(19) United States
(12) Patent Application Publication
(10) Pub. No.: US 2026/0219379 A1
(43) Pub. Date: Jul. 30, 2026

(54) BIOMETRIC DETECTION DEVICE

(71) Applicant: DENSO CORPORATION, Kariya-city (JP)

(72) Inventors: Shunsuke SHIBATA, Nisshin-city (JP); Yoichiro SUZUKI, Nisshin-city (JP); Yuuji KAKUYA, Kariya-city (JP); Kenichiro SANJI, Kariya-city (JP)

(21) Appl. No.: 19/574,783

(22) Filed: Mar. 23, 2026

Related U.S. Application Data
(63) Continuation of application No. PCT/JP2024/031857, filed on Sep. 5, 2024.

(30) Foreign Application Priority Data
Sep. 25, 2023 (JP) 2023-161604

(51) Int. Cl. G01S 13/58; A61B 5/00; A61B 5/05; A61B 5/08

(52) U.S. Cl. CPC G01S 13/581; A61B 5/05; A61B 5/6893; A61B 5/08

(57) ABSTRACT

A biometric detection device for a vehicle includes at least one radar that transmits pulse waves at a predetermined frequency and receives reflected waves. The device further includes a circuit or a processor with a memory storing computer program code. The circuit or processor enables detection of the presence or absence of a biological entity inside the vehicle based on received radar signals. The radar performs multiple transmissions and receptions at predetermined intervals, and the reception time of the reflected waves exceeds the time corresponding to the distance between the radar and the farthest end of the vehicle. Detection is performed by analyzing temporal variations in reception strength at multiple sampling points, including points where indirect waves reflected from the biological entity inside the vehicle are observable.

### CROSS REFERENCE TO RELATED APPLICATIONS

The present application is a continuation application of International Patent Application No. PCT/JP2024/031857 filed on Sep. 5, 2024, which designated the U.S. and claims the benefit of priority from Japanese Patent Application No. 2023-161604, filed on Sep. 25, 2023. The entire disclosures of all of the above applications are incorporated herein by reference.

### TECHNICAL FIELD

The disclosure in this specification relates to a biometric detection device.

### BACKGROUND

An occupant detection device equipped with a radar transmits pulse waves and receives reflected waves.

### SUMMARY

According to at least one embodiment, a biometric detection device mounted on a vehicle includes at least one radar that transmits pulse waves at a predetermined frequency and receives reflected waves of the pulse waves. The device also has at least one of (i) a circuit and (ii) a processor with a memory storing computer program code executable by the processor. The circuit or processor causes the biometric detection device to detect a presence or absence of a biological entity inside the vehicle based on received signals of the radar. The radar may perform transmission and reception multiple times at predetermined intervals. A reception time of the reflected waves may be longer than a time corresponding to a distance between the radar and a position at an end of the vehicle farthest from the radar. The circuit or processor further may cause the biometric detection device to detect the presence or absence of a biological entity based on temporal variations in reception strength at multiple sampling points of the received signals from multiple transmissions and receptions. The sampling points include points where indirect waves reflected from the biological entity inside the vehicle are observable.

### BRIEF DESCRIPTION OF DRAWINGS

The details of one or more embodiments are set forth in the accompanying drawings and the description below. Other features and advantages will be apparent from the description and drawings, and from the claims.

FIG. 1 is a diagram illustrating detection principle.

FIG. 2 is a diagram illustrating an example of a reception waveform by a radar.

FIG. 3 is a diagram illustrating a temporal variation in reception strength of a reflected wave.

FIG. 4 is a diagram illustrating a mounting position of the radar in a vehicle.

FIG. 5 is a diagram illustrating a result of measured reflection intensity.

FIG. 6 is a diagram in which the time shown in FIG. 5 is replaced with distance.

FIG. 7 is a diagram illustrating a temporal variation in the reception strength of a reflected wave when a child is present in a passenger seat.

FIG. 8 is a diagram illustrating a waveform obtained by applying a filtering process to FIG. 7.

FIG. 9 is a diagram illustrating sampling variations in multiple transmission and reception cycles.

FIG. 10 is a diagram illustrating a temporal variation in reception strength of a reflected wave when a child is present in a trunk.

FIG. 11 is a diagram illustrating a waveform obtained by applying a filtering process to FIG. 10.

FIG. 12 is a diagram illustrating a biometric detection device according to the first embodiment.

FIG. 13 is a diagram illustrating an indirect wave passing from a living body via a metal body.

FIG. 14 is a flowchart illustrating a biological detection process.

FIG. 15 is a diagram illustrating a temporal variation of the reception strength at point SP1.

FIG. 16 is a diagram illustrating a waveform obtained by applying filter processing to in FIG. 15.

FIG. 17 is a flowchart illustrating a biological detection process executed by a biometric detection device according to a second embodiment.

FIG. 18 is a flowchart illustrating a biological detection process executed by a biometric detection device according to a third embodiment.

FIG. 19 is a diagram illustrating a modification.

FIG. 20 is a flowchart illustrating a biological detection process executed by a biometric detection device according to a fourth embodiment.

FIG. 21 is a diagram illustrating an example of a displacement of a moving object relative to a radar.

FIG. 22 is a diagram illustrating changes in relative velocity.

FIG. 23 is a diagram illustrating an example of radar arrangement in a biometric detection device according to a fifth embodiment.

FIG. 24 is a flowchart illustrating a biological detection process.

FIG. 25 is a diagram illustrating a received waveform of one pulse for an in-vehicle radar and an external radar.

FIG. 26 is a diagram illustrating an example of installation positions of radars in a vehicle in a biometric detection device according to a sixth embodiment.

FIG. 27 is a diagram illustrating a biological detection process.

### DETAILED DESCRIPTION

To begin with, examples of relevant techniques will be described.

A biometric detection device according to a comparative example equipped with a radar transmits pulse waves and receives reflected waves.

In a case of biometric detection devices that use pulse waves, there is an issue in that detection accuracy of biological subjects is low. For this reason and other reasons that are not described, further improvement is required to be made in a biometric detection device.

In contrast to the comparative example, according to a biometric detection device of the present disclosure, accuracy of detecting biological subjects within a vehicle can be improved.

In a biometric detection device using pulse waves, transmission and reception processes are executed multiple times at predetermined intervals to determine the presence or absence of a biological object. As a result of detailed examination by the inventors, it was found that, during multiple transmission and reception processes, temporal deviations, that is, sampling variations, occur in a received waveform. The variation in the waveform caused by the biological object may be obscured by changes due to sampling variations, making it difficult to accurately detect the biological object. The disclosed biometric detection device is based on this finding.

According to one aspect of the present disclosure, a biometric detection device mounted on a vehicle includes at least one radar that transmits pulse waves at a predetermined frequency and receives reflected waves of the pulse waves. The device also has at least one of (i) a circuit and (ii) a processor with a memory storing computer program code executable by the processor. The circuit or processor causes the biometric detection device to detect a presence or absence of a biological entity inside the vehicle based on received signals of the radar. The radar performs transmission and reception multiple times at predetermined intervals. A reception time of the reflected waves is longer than a time corresponding to a distance between the radar and a position at an end of the vehicle farthest from the radar. The circuit or processor further causes the biometric detection device to detect the presence or absence of a biological entity based on temporal variations in reception strength at multiple sampling points of the received signals from multiple transmissions and receptions. The sampling points include points where indirect waves reflected from the biological entity inside the vehicle are observable.

According to this configuration, transmission and reception are performed multiple times, and the presence or absence of a living body is detected based on the received signals from the multiple transmissions and receptions. By lengthening the reception time, the radar receives indirect waves from the living body that travel via the metal body of the vehicle. In this way, multipath is actively utilized. Even if sampling variations occur, the presence or absence of a living body can be detected by means of the indirect waves, which are received with a delay relative to the direct waves from the metal body. Therefore, the detection accuracy of a living body inside the vehicle can be improved.

Hereinafter, multiple embodiments will be described with reference to the drawings. Duplicate description may be omitted by assigning the same reference numerals to the corresponding elements in each embodiment. In cases where only a part of the configuration is described in each embodiment, the configurations of other portions previously described in other embodiments may be applied to those parts. Further, not only the combinations of the configurations explicitly shown in the description of the respective embodiments, but also the configurations of multiple embodiments can be partially combined even when they are not explicitly shown as long as there is no difficulty in the combination in particular.

### First Embodiment

Euro NCAP (European New Car Assessment Program) decided to add the child presence detection function as an evaluation item for in-vehicle occupant sensing applications starting in 2023, and to make it a scoring item. Euro NCAP further determined that, from 2025 onward, only devices equipped with a function to directly detect the presence of a child will be subject to evaluation.

Therefore, it is necessary to detect biological information such as breathing and body movements throughout an entire vehicle interior in order to determine the presence or absence of a child. Since one of the evaluation conditions requires detection when a blanket is covering the child or when the child is on a child seat, it is difficult to achieve this with cameras, and the use of radio waves is being considered as a promising solution.

A biometric detection device according to the present embodiment detects the presence or absence of a living body inside the vehicle using pulse waves, as described below. The biometric detection device can be applied, for example, to the detection of a child being left behind. The living bodies inside the vehicle that can be detected by the biometric detection device are not limited to children, but may also include other occupants, as well as animals such as dogs and cats.

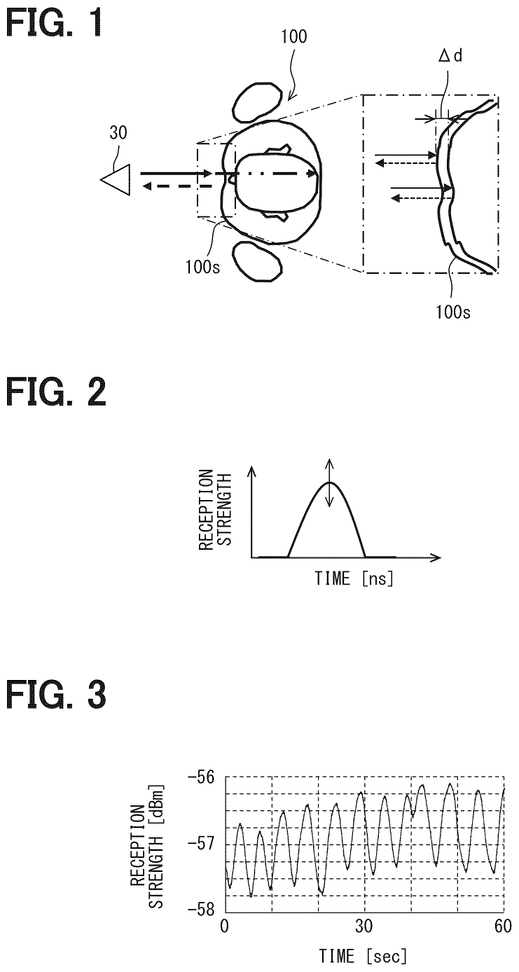

First, with reference to FIG. 1 and FIG. 2, the principle of detecting a living body using a pulse wave as a radio wave will be explained.

### Detection Principle

FIG. 1 illustrates the detection principle. FIG. 1 shows a displacement of a body surface caused by breathing. In FIG. 1, a transmitted pulse wave (transmitted wave) is indicated by a solid arrow, a reflected wave from a human body is indicated by a dashed arrow, and the transmitted wave passing through the human body is indicated by a two-dot chain arrow. FIG. 2 shows an example of a received waveform by a radar. FIG. 3 shows a time variation of the reception strength (received intensity) of the reflected wave. FIG. 3 shows temporal changes in reception strength over multiple transmission and reception cycles. Here, an example of the human body is shown as a living body 100.

As shown in FIG. 1, when a high-frequency pulse wave is emitted from the radar 30 toward the living body 100 (human body), a portion of the wave penetrates the living body 100, while another portion is reflected at a body surface 100s. High frequency refers to, for example, a frequency of 1 GHz or higher. The radar 30 is, for example, a UWB radar. UWB is an abbreviation for Ultra Wide Band. Because the body surface 100s is displaced by breathing, a distance between the radar 30 (antenna) and the living body 100 changes in accordance with respiration. The displacement Δd of the body surface 100s due to breathing is, for example, approximately 1 mm to 10 mm.

Therefore, as indicated by the arrows in FIG. 2, the reception strength of the reflected wave changes in accordance with respiration. FIG. 2 shows a received waveform at a predetermined transmission-reception timing. By monitoring the received waveform for each of multiple transmission-reception cycles, it is possible to obtain a waveform representing the temporal variation in the reception strength of the reflected wave, as shown in FIG. 3. The temporal variation shown in FIG. 3 is characteristic of the respiration of the living body 100. Therefore, the presence or absence of the living body 100 can be detected by using the temporal variation. Similarly, body movement can also be detected by monitoring the received waveform, since the distance between the radar 30 and the living body 100 changes.

### Sampling Variation

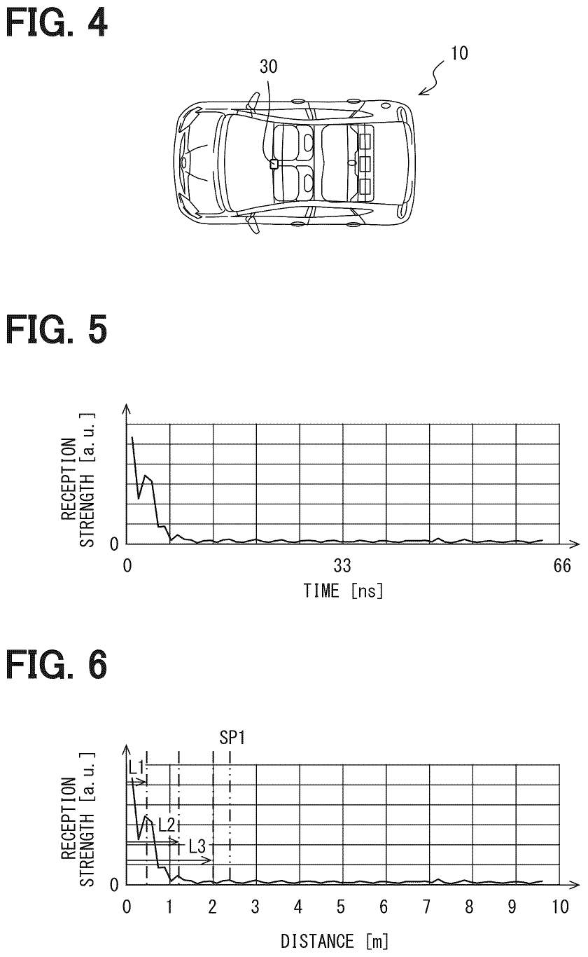

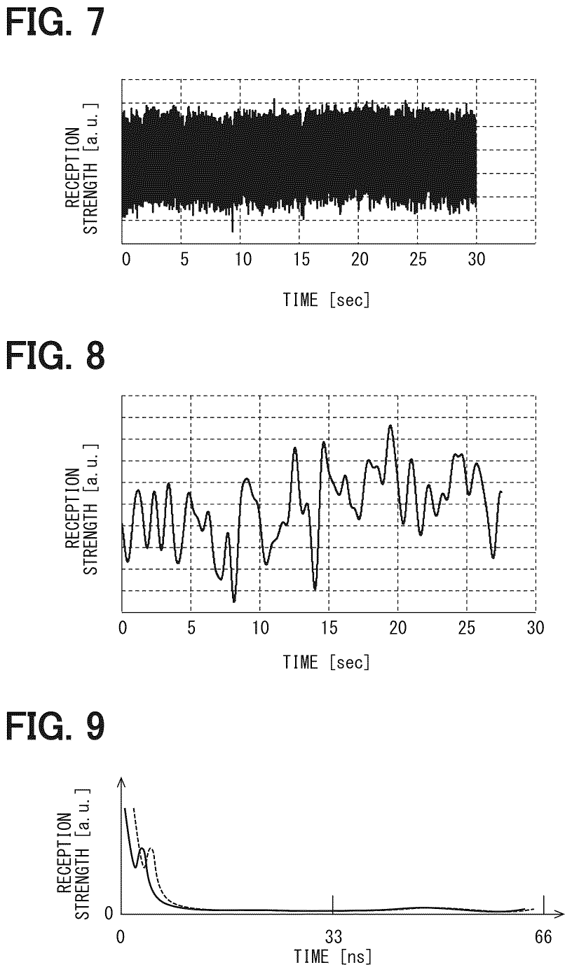

FIG. 4 shows an installation position of the radar (UWB radar) when measuring the reflected intensity. FIG. 5 shows a result of measuring the reflected intensity. FIG. 6 is a diagram in which the time shown in FIG. 5 is replaced with distance. For convenience, FIG. 6 shows a value that is half of a round-trip distance corresponding to the reception strength as the distance, that is, a one-way distance. FIG. 7 shows a time variation of the reception strength of the reflected wave when a child (living body) is present in a passenger seat. FIG. 7 corresponds to FIG. 3. FIG. 7 shows the time variation of the reception strength when the child is present at distance L1. FIG. 8 shows a waveform obtained by applying a filtering processing to FIG. 7. FIG. 8 shows the waveform after frequency filtering (0.1 to 1 Hz). FIG. 9 shows the sampling variation in multiple transmissions and receptions.

As shown in FIG. 4, for measurement of the reflected intensity, the radar 30 is arranged near a front end of a vehicle ceiling and near a center in a left-right direction, specifically in an overhead console. The received waveform in FIG. 5 shows a waveform (baseline waveform) when no living body is present inside the vehicle. A horizontal axis represents an elapsed time from the transmission of the pulse wave to the reception of the reflected wave, that is, a reception time. As shown in FIG. 5, a section where the reception strength is high near time zero (0) is due to reflection (direct wave) from a ceiling metal of the vehicle ceiling. In the received waveform, reflections from not only the ceiling but also a metal body such as a vehicle body are superimposed according to their distance from the radar 30. In regions with short reception times, influence of direct waves from the metal body is significant, resulting in high reception strength.

When detecting the presence or absence of a living body inside the vehicle, for example, the presence or absence of a child, it is conceivable to monitor the reception strength corresponding to a distance between each seat where a child may be present and the radar 30. In this case, as shown in FIG. 6, time is converted to distance, and fluctuations at locations corresponding to the distance from the radar 30 to each seat are detected. The distance (one-way distance) shown in FIG. 6 and the time Δt from transmission to reception of the pulse wave satisfy a relationship expressed by Equation 1 below. “c” is the speed of light (3×10\hat{ }8 m/s). Twice the one-way distance is the round-trip distance.

$$One - way distance \times 2 = c \times Δ t \qquad ( 1 )$$

A dashed lines shown in FIG. 6 indicate positions of front seats (driver's seat, passenger seat), rear seats (behind the driver's seat, behind the passenger seat), and a trunk. The front seats are located at a distance L1 from the radar 30. The rear seats are located at a distance L2 from the radar 30. The trunk is located at a distance L3 from the radar 30. The distances L1, L2, and L3 satisfy a relationship L1<L2<L3. A “D” seat is the driver's seat, and a “P” seat is the passenger's seat.

However, as shown in FIGS. 7 and 8, it was found that even if a child is present in the front seat, waveforms corresponding to breathing cannot be detected. A reason detection cannot be performed at positions close to the radar 30, such as the D seat or P seat, is due to sampling variation of the radar 30. During multiple transmissions and receptions, a time shift on the order of nanoseconds occurs in the received waveform, as illustrated in FIG. 9. Therefore, variations occur in baseline reception strength at the predetermined sampling points.

This variation has a greater impact as the reception strength increases. For example, when a child is present in a seat corresponding to a location where the influence of ceiling reflections remains (such as a front seat), minute fluctuations such as breathing may be buried in the changes in reception strength caused by sampling variation. As described above, it has become apparent that fluctuations caused by a living body may be buried in the changes caused by sampling variation, resulting in the possibility that the living body cannot be detected.

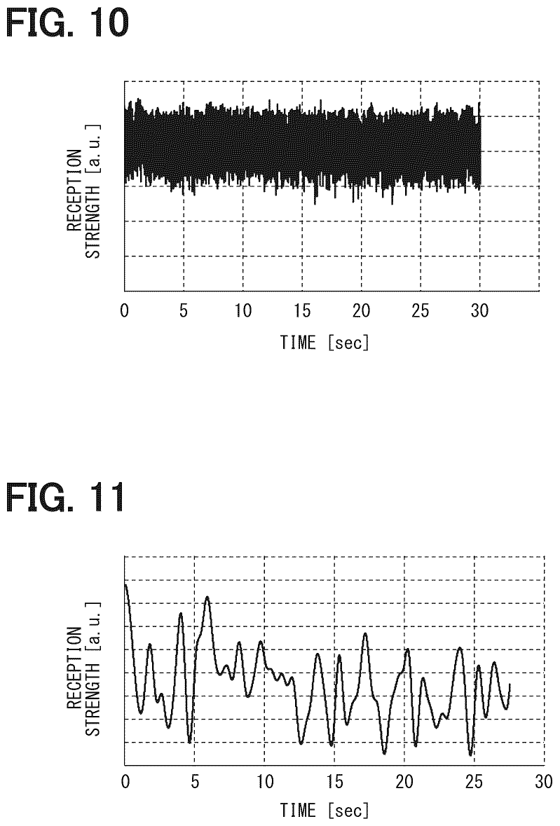

FIG. 10 shows time variation of the reception strength of the reflected wave when a child is present in the trunk. FIG. 10 corresponds to FIG. 3. FIG. 10 shows the time variation of the reception strength when the child is present at distance L3. FIG. 11 shows the waveform after the filtering processing has been applied to FIG. 10. Similar to FIG. 8, FIG. 11 shows the waveform after frequency filtering (0.1 to 1 Hz).

Even when a child is present in the trunk, as shown in FIGS. 10 and 11, a waveform corresponding to breathing could not be detected. The trunk is located at a position distant from the radar 30, and there are obstacles such as seats on a direct path from the radar 30 to the trunk. Therefore, it is considered that the radio waves are attenuated, making it difficult to detect breathing.

The biometric detection device according to the present embodiment is based on the above findings. Next, a schematic configuration of the biometric detection device will be described.

### Biometric Detection Device

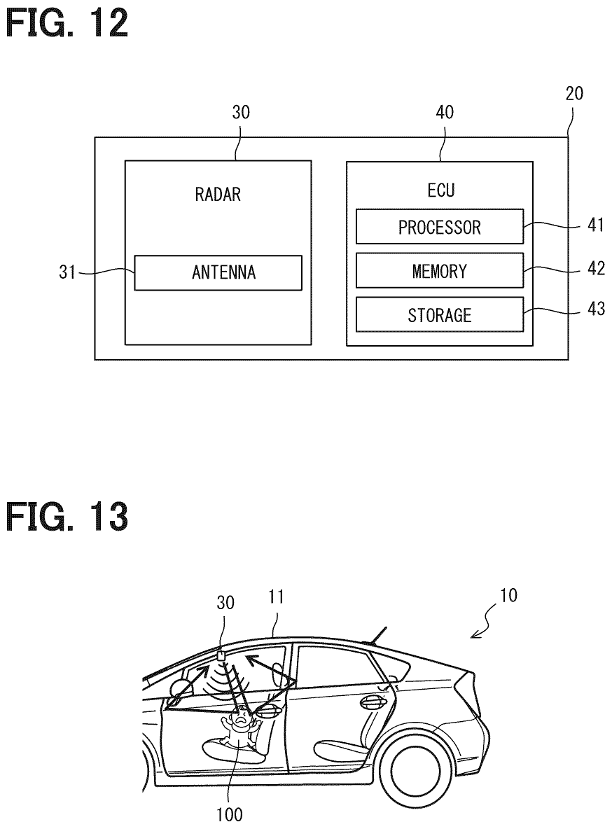

FIG. 12 shows a schematic configuration of the biometric detection device according to the present embodiment. FIG. 13 shows an indirect wave. As shown in FIG. 12, the biometric detection device 20 is provided with at least one radar 30 and an ECU 40. ECU is an abbreviation for Electronic Control Unit.

The radar 30 transmits pulse waves of a predetermined frequency and receives reflected waves of the pulse waves. The radar 30 has an antenna 31. The antenna 31 may include separate antennas for transmission and reception, or it may include a shared antenna used for both transmission and reception. As described above, the frequency of the pulse wave is a high frequency, for example, a frequency of 1 GHz or higher. The radar 30 is a UWB radar or a millimeter wave radar. As one example, the radar 30 of the present embodiment is an IR-type UWB radar. IR is an abbreviation for Impulse Radio. The impulse signal used in UWB communication may be a signal with an extremely short pulse width (for example, 2 ns) and a bandwidth of 500 MHz (strictly speaking, 499.2 MHz) or greater, that is, an ultra-wide bandwidth.

The radar 30 is mounted on a vehicle 10. The biometric detection device 20 may be equipped with only one radar 30, or may be equipped with radars 30. As one example, the biometric detection device 20 of the present embodiment is equipped with only one radar 30. As shown in FIGS. 4 and 13, the radar 30 is disposed on the vehicle interior, near the front end of the ceiling 11, and near the center in the left-right direction. The radar 30 is disposed in the overhead console. The radar 30 may, for example, also serve as a radar for a smart entry system. The antenna 31 may, for example, be an omnidirectional antenna.

During the biometrics detection period described later, the radar 30 executes transmission and reception multiple times at predetermined intervals. The radar 30 performs multiple transmissions and receptions at time intervals that are sufficiently short relative to the breathing cycle. The reception time of the reflected wave is longer than the time corresponding to the distance between the radar 30 and the position farthest from the radar 30 at an end of the vehicle 10. Therefore, as shown in FIG. 13, the radar 30 receives indirect waves from the living body via the metallic body of the vehicle 10. In this way, the biometric detection device 20 actively utilizes multipath. As one example, in the present embodiment, the vehicle 10 is a passenger car with five seats. In the vehicle 10, the end farthest from the radar 30 is a rear end of the vehicle, and a distance from the radar 30 to the rear end of the vehicle is approximately 2 meters. The reception time of the reflected wave is longer than the time corresponding to the 2-meter distance to the rear end of the vehicle. The reception time may be, for example, the time corresponding to the round-trip distance of 4 meters between the radar 30 and the rear end of the vehicle (approximately 13 ns). The radar 30 may perform the next transmission and reception after 100 ms have elapsed following the previous transmission and reception.

The ECU 40 controls operation of the radar 30. The ECU 40 detects the presence or absence of a living being inside the vehicle based on the received signal from the radar 30. The ECU 40 corresponds to a detection unit. The ECU 40 detects the presence or absence of a living being based on multiple received signals. As one example, the ECU 40 of the present embodiment detects the presence or absence of a living being based on an amount of temporal variation in multiple received signals. The ECU 40 is configured to include components such as a processor 41, a memory 42, and a storage 43.

The processor 41 executes various processes by accessing the memory 42. The memory 42 is a rewritable volatile storage medium. The memory 42 is, for example, a RAM. RAM is an abbreviation for Random Access Memory. The storage 43 is a rewritable nonvolatile storage medium. The storage 43 stores a program to be executed by the processor 41. By accessing the memory and executing programs, the ECU 40 constructs multiple functional units. The execution of the program by the processor 41 corresponds to the execution of the biometric detection method described later. The ECU 40 may include multiple processors 41. For example, the ECU 40 may also be shared with an ECU of a smart entry system. The ECU 40 may include, for example, a function for detecting the presence or absence of a living being inside the vehicle together with the radar 30, such as a function for detecting if a child has been left behind.

### Biometric Detection Method

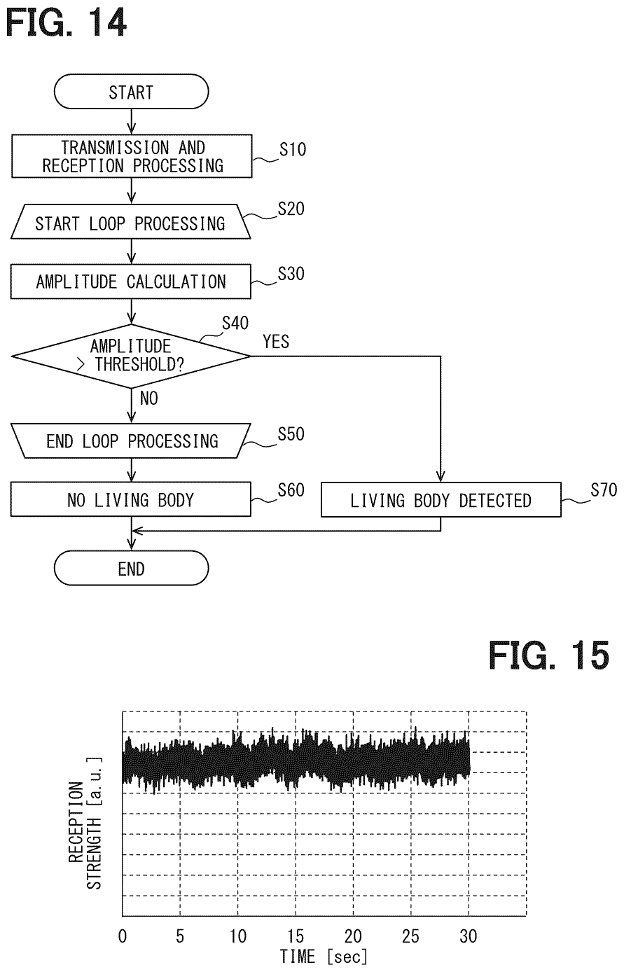

FIG. 14 is a flowchart illustrating processing executed by the biometric detection device, that is, the biometric detection method. When, for example, a door of the vehicle 10 is locked, the biometric detection device 20 executes the biometric detection processing shown in FIG. 14.

The ECU 40 controls the operation of the radar 30 so as to execute transmission and reception processing for a predetermined period (step S10). As a result, the radar 30 executes the transmission and reception processing. In the present embodiment, as an example, a period of 10 seconds from door locking is set as the predetermined period. During the predetermined period, the radar 30 repeatedly performs transmission and reception multiple times at predetermined intervals. As described above, the reception time for one transmission is set to 13 ns, and multiple transmissions and receptions are performed at intervals of 100 ms.

Next, the ECU 40 acquires received signals from the radar 30 during the predetermined period and starts loop processing (Step S20). The ECU 40 sequentially executes the loop processing for each preset sampling point. The sampling points are, for example, predetermined values of distance as shown in FIG. 6, or predetermined values of time as shown in FIG. 5. The sampling points may be set at predetermined intervals of distance or predetermined intervals of time. The sampling points may be set, for example, at intervals of 0.1 m or at intervals of 1 ns. The sampling points may also be set based on data obtained from tests or the like.

The ECU 40 calculates the time variation in reception strength, that is, the amplitude, at predetermined sampling points (step S30). Next, the ECU 40 compares the calculated amplitude with a threshold value and determines whether the amplitude is greater than the threshold (step S40). As described above, the reception strength of the base waveform is generally greater the closer it is to the radar 30. The threshold value is set according to the sampling point. The threshold value is set according to the amplitude of the base waveform. The threshold value is set in consideration of sampling variation, that is, variation in the reception strength of the base waveform. As a result, erroneous determination can be reduced.

When the amplitude is below the threshold value, the ECU 40 executes the processes of steps S30 and S40 for the next sampling point. When the amplitude is below the threshold value for all sampling points, the ECU 40 terminates the loop processing (step S50). Next, the ECU 40 determines that no living body 100 is present inside the vehicle (step S60), and terminates the series of processes. In step S40, when the amplitude is greater than the threshold value, the ECU 40 determines that respiration or body movement is superimposed on the base waveform, in other words, that a living body 100 is present inside the vehicle (step S70), and terminates the series of processes.

The ECU 40 detects the presence or absence of the living body 100 based on temporal variations in the reception strength at the sampling points. As described above, in the sampling of reflected waves immediately after transmission, direct waves from the metal body such as the ceiling 11 are superimposed. Therefore, fluctuations such as those caused by breathing are likely to be buried in the changes in reception strength due to sampling variations.

When sampling the reflected waves after a certain period of time has elapsed following transmission, the influence of the direct wave from the metal body becomes small. At this time, the reflected wave from the living body 100 is multiply reflected within the vehicle body and the waveform that reaches the radar 30 is observed. The indirect wave from the living body 100, which passes via the metal body, reaches the radar 30 with a delay compared to the direct wave from the metal body. Therefore, fluctuations such as those caused by breathing are more likely to appear in areas where the reflected intensity of the base waveform is lower, compared to areas where it is higher.

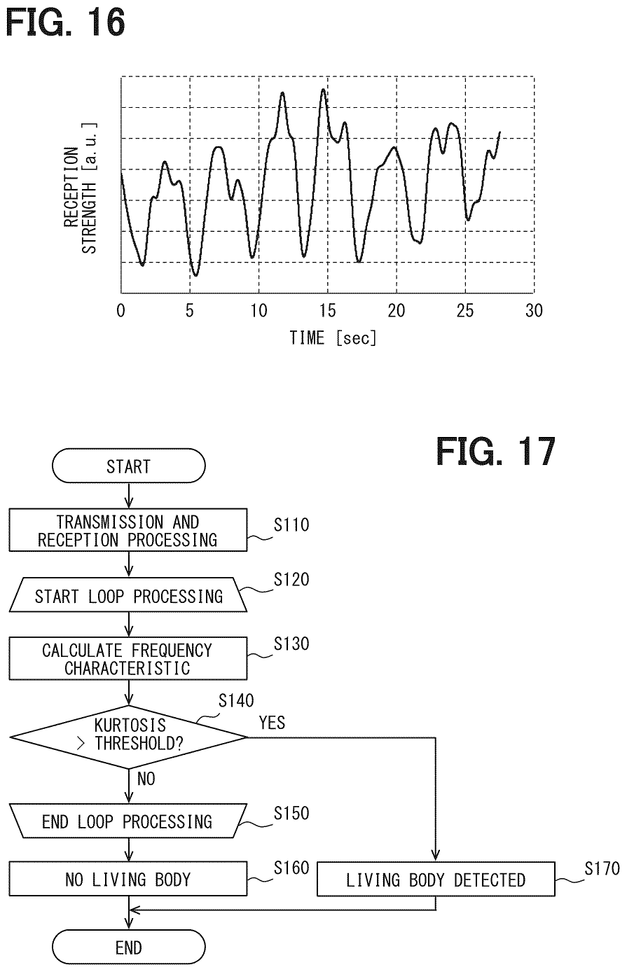

FIG. 15 shows the time variation of the reception strength at sampling point SP1 indicated by a two-dot chain line in FIG. 6. FIG. 15 corresponds to FIG. 3. FIG. 16 shows the waveform after filtering has been applied to the data in FIG. 15. Similar to FIG. 8, FIG. 16 shows the waveform after applying the frequency filter (0.1 to 1 Hz). The sampling point SP1 is located at a distance longer than 2 meters, the distance to the rear end of the vehicle, for example, at 2.4 meters. The distance to sampling point SP1 is greater than twice the distance to the front seat (distance L1). The ECU 40 includes sampling point SP1 as one of its sampling points. As shown in FIG. 16, at sampling point SP1, the influence of the direct wave from the metal body is diminished, and an indirect wave passing from the living body 100 through the metal body is observed. In FIG. 16, fluctuations due to breathing appear as changes in amplitude.

Furthermore, at positions distant from the radar 30, such as the trunk, the direct wave may be attenuated as described above, and it is possible that it does not reach the living body 100 present in the trunk. However, via reflection paths such as a floor in the vehicle, the pulse wave can reach the living body 100, and the reflected wave from the living body 100 may undergo multiple reflections within the vehicle body and reach the radar 30. Therefore, as described above, at sampling points where the influence of the direct wave from the metal body is diminished, it is possible to detect fluctuations caused by breathing or the like.

### Summary of First Embodiment

In the present embodiment, attention is focused on the fact that reflected waves striking the living body (occupant) are superimposed on the received waveform of the radar 30 while being delayed in the time domain along various paths within the vehicle interior. According to the biometric detection device 20 of the present embodiment, the radar 30 performs transmission and reception multiple times, and the ECU 40 (detection unit) detects the presence or absence of the living body 100 based on multiple received signals. In each transmission and reception, the reception time of the reflected wave is longer than the time corresponding to the distance between the radar 30 and the position farthest from the radar 30 at the end of the vehicle 10. By lengthening the reception time, the radar 30 receives indirect waves from the living body 100 that travel via the metal body of the vehicle 10. In this way, multipath is actively utilized.

At sampling points where indirect waves are superimposed, the influence of direct waves from the metal body is small, and for example, the reception strength of the base waveform is low. Therefore, as illustrated in FIG. 9, even if there is a time shift on the order of nanoseconds, that is, sampling variation, over multiple receptions, it is still possible to detect the presence or absence of the living body 100. Therefore, the detection accuracy of the living body 100 inside the vehicle can be improved. For example, the accuracy of detecting a child left behind can be improved.

The ECU 40 may detect the presence or absence of the living body 100 based on the temporal variation in reception strength, that is, the amplitude, across multiple receptions. Variations such as breathing and body movements are superimposed on the base waveform. That is, the amplitude increases due to breathing and body movements. Therefore, the presence or absence of the living body 100 can be detected based on the amplitude. By using amplitude, not only breathing but also variations due to body movement can be detected.

An ultra-wideband (UWB) radar may be used as the radar 30. In recent years, there has been a growing market demand for utilizing smartphones as car keys. In UWB, which is one such method, a position of a smartphone is identified by measuring a communication time between the smartphone and the vehicle using compressed pulse-shaped radio waves, and this is used to trigger unlocking or locking of the vehicle and starting engine. By using a UWB radar, it is possible to combine (share) it with the smart entry system, thereby reducing costs. There is no need to install a separate radar for sensing. It is also possible to use a millimeter-wave radar instead of a UWB radar.

### Second Embodiment

The present embodiment is a modified example based on the preceding embodiment and can incorporate the contents of the preceding embodiment. In the first embodiment, the presence or absence of a living body is detected based on amplitude. Alternatively, the presence or absence of a living body may be detected based on frequency characteristics.

FIG. 17 illustrates processing executed by the biometric detection device according to a second embodiment, that is, a biometric detection method. In the biometric detection processing shown in FIG. 17, the processing of steps S130 and S140 differs from the processing of steps S30 and S40 shown in FIG. 14. The other processes are the same as those shown in FIG. 14.

For example, when the door of the vehicle 10 is locked, the biometric detection device 20 executes the biometric detection processing shown in FIG. 17. The processing of steps S110 and S120 is the same as the processing of steps S10 and S20 shown in FIG. 14. In the loop processing, the ECU 40 calculates frequency characteristics (step S130). For example, the ECU 40 calculates the frequency characteristics based on the temporal variation of the reception strength at predetermined sampling points. The ECU 40 uses fast Fourier transform (FFT) on the temporal variation (amplitude) of the reception strength to calculate the frequency characteristics. The ECU 40 calculates, for example, kurtosis as the frequency characteristic. Kurtosis is obtained by dividing the reception strength at a peak frequency by average strength at other frequencies.

Next, the ECU 40 compares the calculated kurtosis with a threshold value and determines whether the kurtosis is greater than the threshold (step S140). When the kurtosis is equal to or less than the threshold, the ECU 40 executes the processing of steps S130 and S140 for the next sampling point. When the kurtosis is equal to or less than the threshold at all sampling points, the ECU 40 terminates the loop processing (step S150), determines that no living body 100 is present in the vehicle interior (step S160), and ends the series of processes. The processing in step S150 is similar to the processing in step S50. The processing in step S160 is similar to the processing in step S60.

When, in step S140, the kurtosis is greater than the threshold, the ECU 40 detects breathing, that is, determines that a living body 100 is present in the vehicle interior (step S170), and ends the series of processes. The processing in step S170 is similar to the processing in step S70. Other configurations are the same as the configurations described in the first embodiment.

### Summary of Second Embodiment

According to the second embodiment, the same effects as those of the first embodiment can be achieved. For example, even if there is a time shift on the order of nanoseconds, that is, sampling variation, over multiple receptions, it is still possible to detect the presence or absence of the living body 100. Therefore, the detection accuracy of the living body 100 inside the vehicle can be improved.

The ECU 40 may detect the presence or absence of the living body 100 based on the frequency characteristics of the received signals obtained multiple times. The presence or absence of the living body 100 can be detected based on whether the frequency characteristics are specific to respiration.

### Third Embodiment

A third embodiment is a modified example based on the basic form of the preceding embodiment, and the description of the preceding embodiment can be incorporated by reference. In the preceding embodiment, the presence or absence of a living body was detected based on amplitude or frequency characteristics. Alternatively, the presence or absence of a living body may be detected based on both amplitude and frequency characteristics.

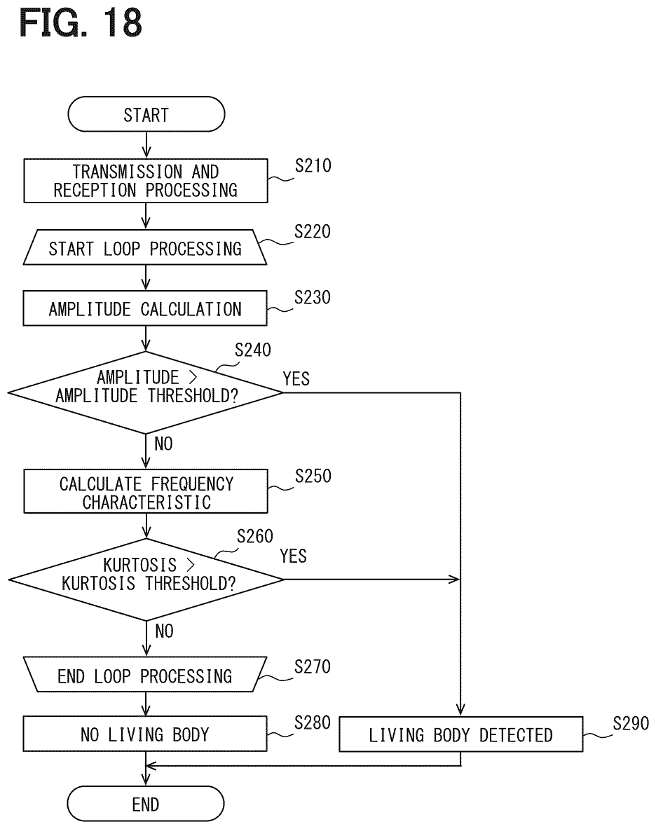

FIG. 18 illustrates processing executed by a biometric detection device according to the present embodiment, that is, a biometric detection method. The biometric detection processing shown in FIG. 18 is configured by adding steps S130 and S140 of FIG. 17 to the processing illustrated in FIG. 14.

The biometric detection device 20 executes the biometric detection processing shown in FIG. 18, for example, when the door of the vehicle 10 is locked. The processing of steps S210, S220, S230, and S240 is the same as the processing of steps S10, S20, S30, and S40 shown in FIG. 14. When the amplitude is equal to or less than the amplitude threshold in step S240, the ECU 40 calculates the frequency characteristics (step S250). The ECU 40 calculates, for example, the kurtosis as the frequency characteristic. Next, the ECU 40 determines whether the kurtosis is greater than the kurtosis threshold (step S260). The processing of steps S250 and S260 is the same as the processing of steps S130 and S140 shown in FIG. 17.

When the kurtosis is equal to or less than the threshold, the ECU 40 executes the processing from step S230 onward for the next sampling point. When, for all sampling points, the amplitude is equal to or less than the amplitude threshold and the kurtosis is equal to or less than the kurtosis threshold, the ECU 40 terminates the loop processing (step S270), determines that there is no living body 100 present in the vehicle interior (step S280), and ends the series of processing. The processing of steps S270 and S280 is the same as the processing of steps S50 and S60.

When, in step S240, the amplitude is greater than the amplitude threshold, the ECU 40 determines that a living body 100 is present in the vehicle interior (step S290) and ends the series of processing. When, in step S260, the kurtosis is greater than the kurtosis threshold, the processing of step S290 is also executed, and the series of processing is terminated. The processing in step S290 is similar to the processing in step S70. Other configurations are the same as the configurations described in the first embodiment.

### Summary of Third Embodiment

According to the third embodiment, the same effects as those of the first embodiment can be achieved. For example, even if there is a time shift on the order of nanoseconds, that is, sampling variation, over multiple receptions, it is still possible to detect the presence or absence of the living body 100. Therefore, the detection accuracy of the living body 100 inside the vehicle can be improved.

The ECU 40 first determines the presence or absence of the living body 100 based on time variations, that is, amplitudes, of received signals. When the living body 100 is not detected based on the amplitudes, the ECU 40 may then determine the presence or absence of the living body 100 based on the frequency characteristics of the received signals. By using amplitude as described above, it is possible to detect not only fluctuations caused by breathing of the living body 100, but also fluctuations caused by body movements. Furthermore, even if minute fluctuations due to breathing are obscured by variations in the received signal caused by sampling variation and the living body 100 cannot be detected by amplitude, breathing can still be detected, that is, the living body 100 can be detected, by using frequency characteristics. In other words, the detection accuracy of the living body 100 inside the vehicle can be further improved.

### Modifications

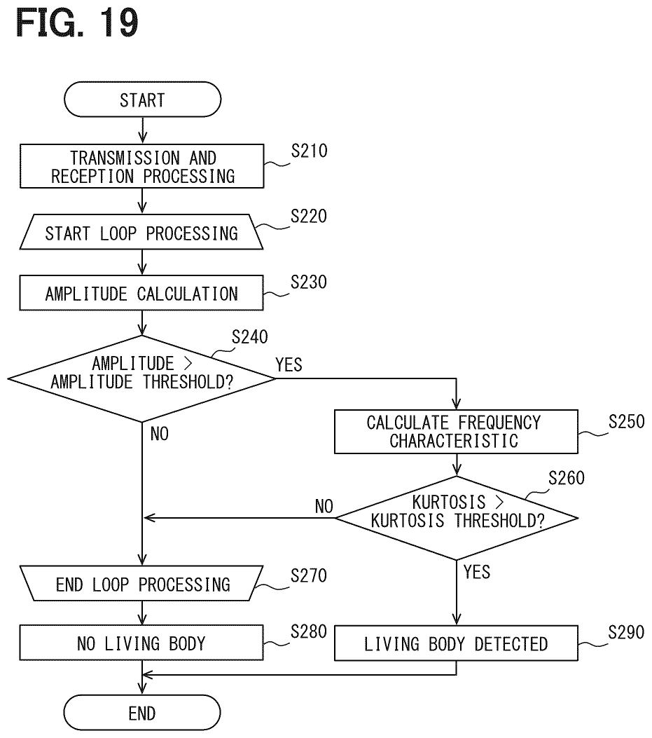

The ECU 40 first detects the presence or absence of the living body 100 based on amplitude, and when the living body 100 is detected based on amplitude, it may further detect the presence or absence of the living body 100 based on the frequency characteristics of the received signals. As shown in FIG. 19, the ECU 40 determines whether the amplitude is greater than the amplitude threshold (step S240), and when the amplitude is greater than the amplitude threshold, calculates the frequency characteristics (step S250). The ECU 40 determines whether the kurtosis is greater than the kurtosis threshold (step S260), and when the kurtosis is greater than the kurtosis threshold, determines that a living body is present (step S290). In other words, it is determined that a living body is present when the amplitude is greater than the amplitude threshold and the kurtosis is greater than the kurtosis threshold. When the values are below the threshold in steps S240 or S260, the loop process is executed until there are no remaining sampling points, and when the sampling points are exhausted, the loop process is terminated (step S270) and it is determined that no living body is present (step S280).

Increasing the number of sampling points raises the likelihood of detecting the living body 100, however, it also increases the possibility of responding to disturbances such as pedestrians outside the vehicle. When using amplitude, for example, when the radar 30 is mounted on the ceiling, there may be cases where the minute fluctuation in respiration at a position far from the radar 30, such as at the feet, is equivalent to the fluctuation in respiration outside the vehicle. According to the present embodiment, not only amplitude but also frequency characteristics are used in combination. Since the presence or absence of the living body 100 is detected in two stages, false detections can be reduced.

### Fourth Embodiment

A fourth embodiment is a modification based on the basic form of the preceding embodiment, and the description of the preceding embodiment can be incorporated by reference. In the preceding embodiment, an example was shown in which disturbances are eliminated by using amplitude and frequency characteristics. Alternatively, or additionally, disturbances may be eliminated by using relative velocity.

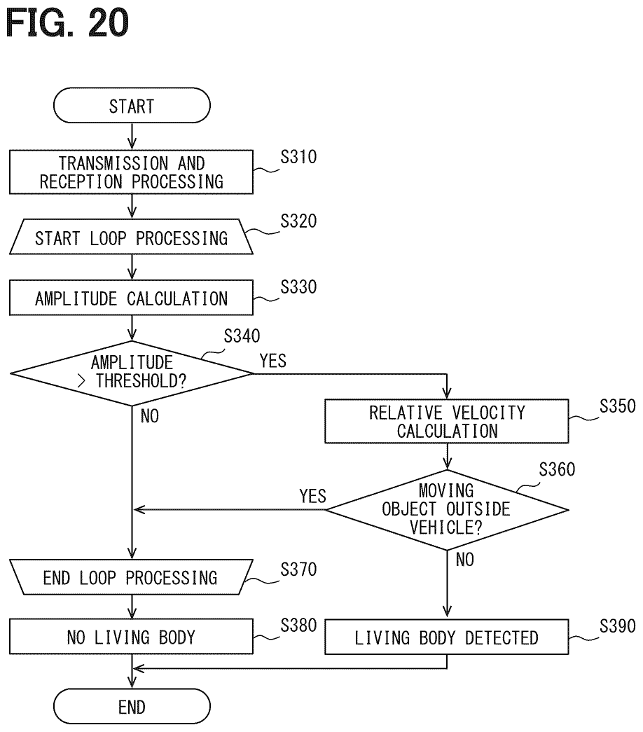

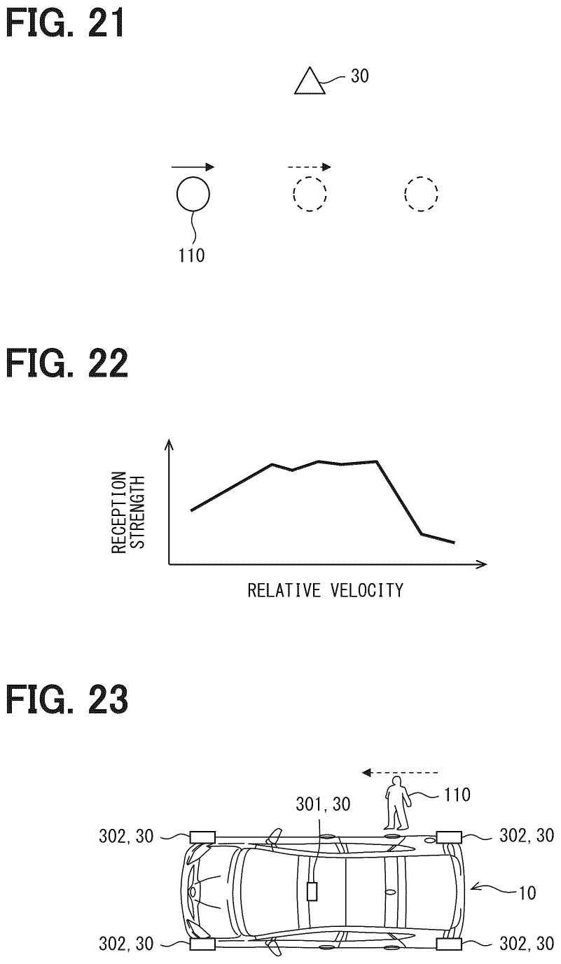

FIG. 20 illustrates processing executed by a biometric detection device according to the fourth embodiment, that is, a biometric detection method. FIG. 21 illustrates an example of a displacement of a moving object with respect to a radar (for example, a UWB radar). FIG. 22 illustrates the change in relative velocity.

The biometric detection device 20 executes the biometric detection processing shown in FIG. 20, for example, when the door of the vehicle 10 is locked. The processing of steps S310, S320, S330, and S340 is the same as that of steps S10, S20, S30, and S40 shown in FIG. 14.

When the amplitude is greater than the threshold value in step S340, the ECU 40 calculates the relative velocity based on any one of the received waveforms (received signals) among the received signals (received waveforms) (step S350). The ECU 40 determines whether the detected object is a moving object 110 located outside the vehicle based on the relative velocity (step S360).

By using the radar 30 as a Doppler sensor, it is possible to calculate the relative velocity of the moving object 110 with respect to the radar 30. When calculating the relative velocity using the Doppler principle, a time window used for measurement, that is, the reception time of the reflected wave, is made longer. As a result, the calculated value includes components of the relative velocity that occurred during that time window. As shown in FIG. 21, when the moving object 110 moves so as to cross in front of the radar 30, widening the time window results in obtaining the component of relative velocity in a line-of-sight direction, which consequently changes from moment to moment. As a result, as shown in FIG. 22, various values are obtained as the relative velocity. This can be used as a feature quantity to determine whether the moving object 110 is outside the vehicle or not. The moving object 110 outside the vehicle may be, for example, a pedestrian outside the vehicle or an adjacent vehicle.

In addition, a distance to the moving object 110 may be calculated together with the relative velocity, and the presence or absence of the moving object 110 outside the vehicle may be determined based on both the distance and the relative velocity. By adding the distance, it is possible to more reliably detect whether the moving object 110 is outside the vehicle.

When the amplitude is below the threshold in step S340, or when it is determined to be the moving object 110 outside the vehicle in step S360, the ECU 40 executes the loop processing until there are no more sampling points. When there are no more sampling points, the loop processing ends (step S370), and it is determined that there is no living body present (step S380). The processing in steps S370 and S380 is the same as the processing in steps S50 and S60.

When it is determined in step S360 that the moving object 110 is not outside the vehicle, the ECU 40 determines that a living body is present (step S390). In other words, it is determined that a living body is present when the amplitude is greater than the threshold and the moving object 110 is not outside the vehicle. The processing in step S390 is similar to the processing in step S70. Other configurations are the same as the configurations described in the first embodiment.

### Summary of Fourth Embodiment

According to the fourth embodiment, the same effects as those of the first embodiment can be achieved. For example, even if there is a time shift on the order of nanoseconds, that is, sampling variation, over multiple receptions, it is still possible to detect the presence or absence of the living body 100. Therefore, the detection accuracy of the living body 100 inside the vehicle can be improved.

When detecting the presence or absence of the living body 100 inside the vehicle, the ECU 40 may calculate the relative speed of the moving object 110 based on any received signal and determine whether the moving object 110 is present outside the vehicle. By using the relative speed, it is possible to determine whether the living body 100 detected based on amplitude is the moving object 110 outside the vehicle. By using the relative velocity, disturbances such as pedestrians outside the vehicle or adjacent vehicles can be eliminated, thereby reducing false detections. In other words, the detection accuracy of the presence or absence of the living body 100 inside the vehicle can be improved.

An example has been shown in which detection of the living body 100 using amplitude is combined with detection of the moving object 110 outside the vehicle using relative speed (relative velocity), however, the present invention is not limited thereto. Detection of the living body 100 using frequency characteristics may also be combined with detection of the moving object 110 outside the vehicle using relative speed. Detection of the living body 100 using amplitude and frequency characteristics may also be combined with detection of the moving object 110 outside the vehicle using relative speed.

### Fifth Embodiment

A fifth embodiment is a modification based on the basic form of the preceding embodiment, and the description of the preceding embodiment can be incorporated by reference. In the preceding embodiment, the biometric detection device is equipped with only one radar disposed inside the vehicle. Alternatively, a device may be provided with a radar disposed inside the vehicle and a radar disposed outside the vehicle.

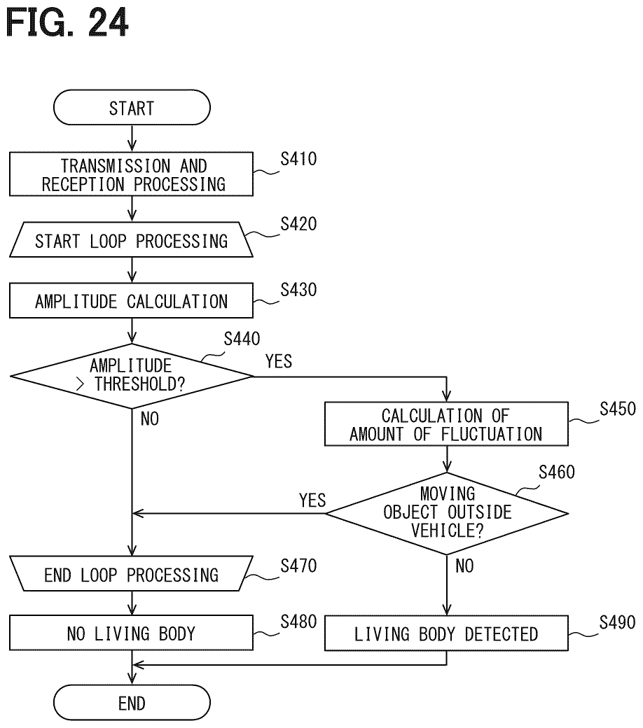

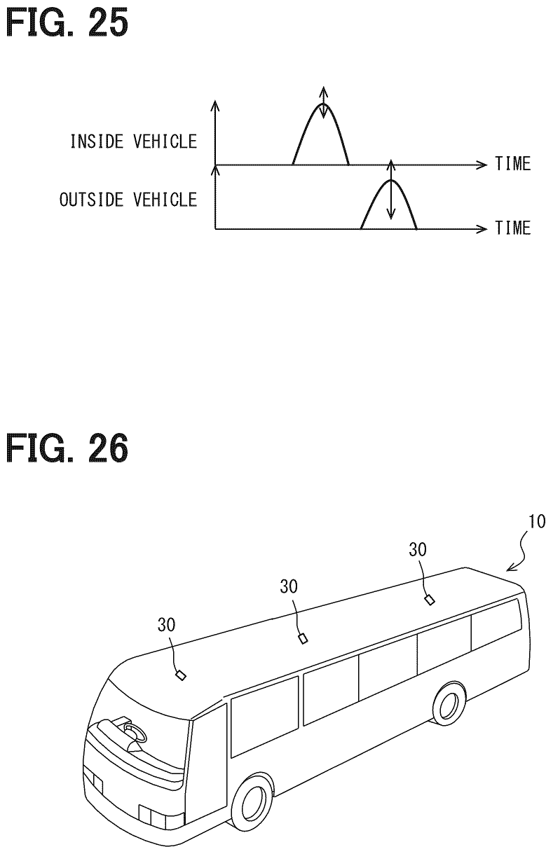

FIG. 23 shows an example of radar arrangement in a biometric detection device according to the present embodiment. FIG. 24 illustrates processing executed by the biometric detection device, that is, a biological detection method. FIG. 25 shows a received waveform of one pulse for an in-vehicle radar and an external radar.

As exemplified in FIG. 23, the biometric detection device 20 is provided with, as the radar 30, the in-vehicle radar 301 and the out-of-vehicle radar 302. The in-vehicle radar 301 is a radar 30 disposed inside the vehicle, and the out-of-vehicle radar 302 is a radar 30 disposed outside the vehicle. In the present embodiment, as an example, the radars 30 are also used in common with the smart entry system. The radars 30 are, for example, UWB radars. The radars 30 include one in-vehicle radar 301 and four out-of-vehicle radars 302. The in-vehicle radar 301 is used, for example, for engine start. The out-of-vehicle radars 302 are arranged near four corners of the vehicle 10. The out-of-vehicle radars 302 are used for locking and unlocking the key.

The biometric detection device 20 executes the biometric detection processing shown in FIG. 24, for example, when the door of the vehicle 10 is locked. The biometric detection device 20 controls the operation of the radars 30 (301, 302) so that the transmission and reception process is performed for a predetermined period (Step S410). The ECU 40 operates the out-of-vehicle radar 302 in parallel with the in-vehicle radar 301. The ECU 40 controls, for example, the in-vehicle radar 301 and the out-of-vehicle radar 302 so that the pulse transmission timing, the reception timing of the reflected waves, and the interval between transmission and reception are approximately synchronized. The processes of steps S410, S420, S430, and S440 are similar to the processes of steps S10, S20, S30, and S40 shown in FIG. 14. The ECU 40 executes steps S430 and S440 based on the received signal from the in-vehicle radar 301. When the amplitude in step S440 is greater than the threshold, the ECU 40 calculates a variation amount in the reception strength caused by the moving object 110 (step S450). The ECU 40 determines whether the object is an external moving object 110 based on magnitude relationship of the variation amount (step S460).

As described above, the biometric detection device 20 is equipped with the in-vehicle radar 301 and the out-of-vehicle radar 302. Disturbances caused by external moving objects 110 can be eliminated based on the difference between the reception strength of the in-vehicle radar 301 and that of the out-of-vehicle radar 302. For example, when a moving object 110 such as a pedestrian or an adjacent vehicle crosses outside the vehicle, as indicated by an arrows in FIG. 25, the fluctuation in the reception strength of the in-vehicle radar 301 is small, while the fluctuation in the reception strength of the out-of-vehicle radar 302 becomes large. Therefore, by utilizing this difference, it is possible to determine whether the object is an external moving object 110. For example, when the variation amount in the out-of-vehicle radar 302 is greater than that of the in-vehicle radar 301, the ECU 40 determines that the object is an external moving object 110.

When the amplitude is below the threshold in step S440, or when it is determined to be an external moving object 110 in step S460, the ECU 40 executes the loop processing until there are no more sampling points. When there are no more sampling points, the loop processing ends (step S470), and it is determined that there is no living body present (step S480). The processing in steps S470 and S480 is the same as the processing in steps S50 and S60.

When it is determined in step S460 that the moving object 110 is not outside the vehicle, the ECU 40 determines that a living body is present (step S490). In other words, it is determined that a living body is present when the amplitude is greater than the threshold and the moving object 110 is not outside the vehicle. The processing in step S490 is similar to the processing in step S70. Other configurations are the same as the configurations described in the first embodiment.

### Summary of Fifth Embodiment

According to the fifth embodiment, the same effects as those of the first embodiment can be achieved. For example, even if there is a time shift on the order of nanoseconds, that is, sampling variation, over multiple receptions, it is still possible to detect the presence or absence of the living body 100. Therefore, the detection accuracy of the living body 100 inside the vehicle can be improved.

The radar 30 may include the in-vehicle radar 301 and the out-of-vehicle radar 302. The ECU 40 may detect the presence or absence of the living body 100 based on the received signals from the in-vehicle radar 301 and the out-of-vehicle radar 302. The out-of-vehicle radar 302 is more susceptible to the influence of external moving objects 110, such as pedestrians outside the vehicle or adjacent vehicles, compared to the in-vehicle radar 301. By using the received signals from the in-vehicle radar 301 and the out-of-vehicle radar 302, it is possible to eliminate disturbances such as pedestrians outside the vehicle and adjacent vehicles, thereby reducing false detection. In other words, the detection accuracy of the presence or absence of the living body 100 inside the vehicle can be improved.

An example has been shown in which detection of external moving objects 110 using the received signals from the in-vehicle radar 301 and the out-of-vehicle radar 302 is combined with detection of the living body 100 using amplitude, however, the invention is not limited thereto. Detection of external moving objects 110 using the received signals from the in-vehicle radar 301 and the out-of-vehicle radar 302 may also be combined with detection of the living body 100 using frequency characteristics. Detection of external moving objects 110 using the received signals from the in-vehicle radar 301 and the out-of-vehicle radar 302 may also be combined with detection of the living body 100 using amplitude and frequency characteristics.

### Sixth Embodiment

A sixth embodiment is a modification based on the basic form of the preceding embodiment, and the description of the preceding embodiment can be incorporated by reference. In the preceding embodiment, the biometric detection device is equipped with only one radar disposed inside the vehicle. Alternatively, radars arranged inside the vehicle may be provided.

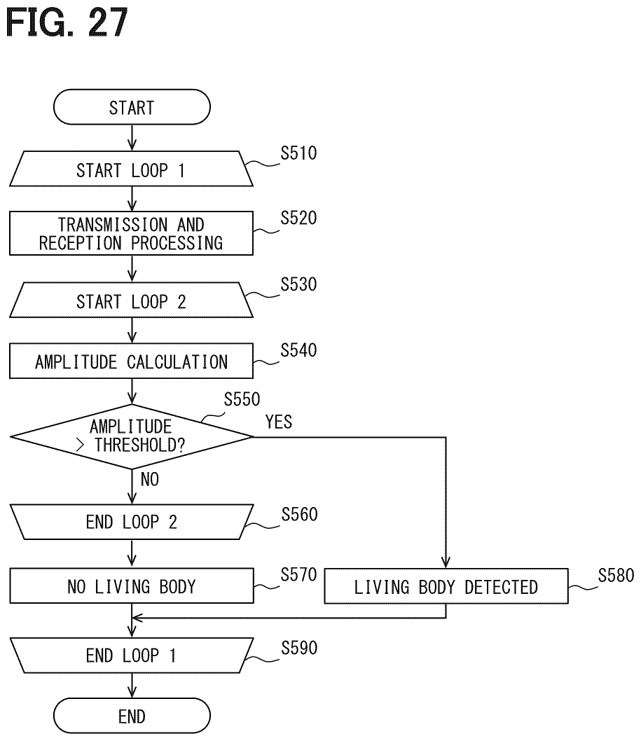

FIG. 26 shows an example of installation positions for multiple radars in a vehicle. FIG. 27 illustrates processing executed by a biometric detection device according to the sixth embodiment, that is, a biological detection method.

As illustrated in FIG. 26, the biometric detection device 20 is provided with radars 30 arranged inside the vehicle. The radars 30 may be, for example, UWB radars. The radars 30 are arranged in a row with a predetermined interval in a front-rear direction of the vehicle 10. Each radar 30 is mounted on a ceiling. In the present embodiment, as an example, the vehicle 10 is a bus. Three radars 30 are mounted on the vehicle 10.

The biometric detection device 20 executes the biometric detection processing shown in FIG. 27, for example, when the door of the vehicle 10 is locked. First, the ECU 40 starts loop process 1 (step S510). The biometric detection device 20 sequentially executes loop processing for each of the radars 30. Next, the ECU 40 controls the operation of the radars 30 to execute transmission and reception processing for a predetermined period (step S520). The ECU 40 acquires received signals from the radars 30 during the predetermined period and starts loop process 2 (step S530). Steps S520, S530, S540, S550, S560, S570, and S580 are the same as the processes in steps S10, S20, S30, S40, S50, S60, and S70 shown in FIG. 14. The loop process 2 (loop 2) corresponds to the loop process described in the preceding embodiment.

When the process of step S570 or step S580 is executed for one of the radars 30, the ECU 40 executes the biometric detection processing from step S520 onward for another one of the radars 30. When the biometric detection processing has been completed for all of the radars 30, the ECU 40 terminates the loop process (step S590) and ends the series of operations. Other configurations are the same as the configurations described in the first embodiment.

### Summary of Sixth Embodiment

According to the sixth embodiment, the same effects as those of the preceding embodiments can be achieved. For example, even if there is a time shift on the order of nanoseconds, that is, sampling variation, over multiple receptions, it is still possible to detect the presence or absence of the living body 100. Therefore, the detection accuracy of the living body 100 inside the vehicle can be improved.

The biometric detection device 20 may include the radars 30 arranged inside the vehicle. The radars 30 may transmit and receive multiple times at mutually different timings, and the ECU 40 may detect the presence or absence of the living body 100 based on the received signals from the multiple transmissions and receptions of each radar 30. In a case of a large vehicle 10, such as a bus, it is envisioned that radars 30 will be installed inside the vehicle to detect the presence or absence of the living body 100. In this case, when a direct wave enters directly from one radar 30 to another radar 30, the reception strength becomes large as described above, and there is a risk that fluctuations caused by the living body 100 may be buried within the fluctuations in reception strength caused by sampling variations. In the present embodiment, since the radars 30 perform multiple transmissions and receptions at mutually different timings, each radar 30 does not receive direct waves from the other radars 30. Therefore, while detecting the presence or absence of the living body 100 within a spacious vehicle interior, it is possible to reduce a decline in detection accuracy of the living body 100 due to the influence of other radars 30. For example, the accuracy of detecting a child left behind can be improved.

A method for reducing or eliminating the influence of direct waves from other radars 30 is not limited to the method of shifting transmission and reception timing illustrated in FIG. 27. For example, a method for removing received direct waves based on the predetermined positional relationship of the antennas may be employed.

The configuration described in this embodiment can be combined with any of the preceding configurations.

### Other Embodiments

The disclosure in this description, the drawings, and the like is not limited to the exemplified embodiments. The disclosure includes exemplary embodiments and modifications by those skilled in the art based on the exemplary embodiments. For example, the disclosure is not limited to the combinations of components and/or elements shown in the embodiments. The disclosure may be implemented in various combinations. The disclosure may include additional portions that can be added to the embodiments. The disclosure includes those in which the components and/or elements of the embodiments are omitted. The disclosure includes the replacement or combination of components and/or elements between one embodiment and another embodiment. The disclosed technical scope is not limited to the description of the embodiments. The several technical scopes disclosed are indicated by the description of the claims, and should be further understood to include meanings equivalent to the description of the claims and all modifications within the scope.

The disclosure in the specification, drawings and the like is not limited by the description of the claims. The disclosures in the description, the drawings, and the like encompass the technical ideas described in the claims, and further extend to a wider variety of technical ideas than those in the claims. Therefore, various technical ideas can be extracted from the disclosure of the specification, the drawings and the like without being limited to the description of the claims.

When an element or layer is referred to as being “on,” “coupled,” “connected,” or “combined,” it may be directly on, coupled, connected, or combined to the other element or layer, or further, intervening elements or layers may be present. In contrast, when an element or a layer is described as “disposed directly above” or “directly connected”, an intervening element or an intervening layer is not present. Other terms used to describe the relationships between elements (for example, “between” vs. “directly between”, and “adjacent” vs. “directly adjacent”) should be interpreted similarly. As used herein, the term “and/or” includes any combination and all combinations relating to one or more of the related listed items. For example, the term A and/or B includes only A, only B, or both A and B. The description of A and/or B means at least one of A and B.

Spatially relative terms such as “inner,” “outer,” “back,” “below,” “low,” “above,” and “high” are utilized herein to facilitate description of one element or feature's relationship to another element(s) or feature(s) as illustrated. Spatial relative terms can be intended to include different orientations of a device in use or operation, in addition to the orientations depicted in the drawings. For example, when the device in the figure is flipped over, an element described as “below” or “directly below” another element or feature is directed “above” the other element or feature. Therefore, the term “below” can include both above and below. The device may be oriented in another direction (rotated 90 degrees or in any other direction) and the spatially relative terms used herein are interpreted accordingly.

The processor 41 may be implemented using a CPU, MPU, GPU, DFP, or the like. “CPU” is an abbreviation for “Central Processing Unit”. “MPU” is an abbreviation for Micro-Processing Unit. “GPU” is an abbreviation for Graphics Processing Unit. “DFP” is an abbreviation for Data Flow Processor.

A part or all of the functions of the processor 41 may be realized by combining multiple types of arithmetic processing devices. A part or all of the functions of the processor 41 may be implemented using an SoC, ASIC, FPGA, or the like. “SoC” is an abbreviation for System-on Chip. “ASIC” is an abbreviation for Application Specific Integrated Circuit. “FPGA” is an abbreviation for Field-Programmable Gate Array. A part or all of the functions of the processor 41 may be implemented using hardware logic circuits.

The program may be stored in a computer-readable non-transitory tangible storage medium as an instruction executed by a computer. As the program storage medium, an HDD, an SSD, a flash memory, or the like can be adopted. “HDD” is an abbreviation of a hard disk drive. “SSD” is an abbreviation for Solid State Drive.

In the above embodiment, an example is shown in which the presence or absence of a living body is detected using intensity obtained from the IQ signal, but the invention is not limited thereto. For example, the presence or absence of a living body may be detected using the IQ signal itself, or by using a phase obtained from the IQ signal. The phase corresponds, for example, to an output value of the arctangent function with a ratio of the Q (Quadrature-Phase) component to the I (In-Phase) component of the received signal as the input value. The magnitude of the I component corresponds to the strength of the in-phase component of the received signal. The magnitude of the Q component corresponds to the strength of the quadrature component of the received signal. The I component is obtained by multiplying the received signal by the carrier wave output from the local oscillator. The Q component is obtained by multiplying the received signal by a signal obtained by shifting the phase of the output signal of the local oscillator by 90°. Furthermore, the presence or absence of a living body may be detected using only the I component of the IQ signal, or using only the Q component.

While the present disclosure has been described with reference to embodiments thereof, it is to be understood that the disclosure is not limited to the embodiments and constructions. To the contrary, the present disclosure is intended to cover various modification and equivalent arrangements. In addition, while the various elements are shown in various combinations and configurations, which are exemplary, other combinations and configurations, including more, less or only a single element, are also within the spirit and scope of the present disclosure.

What is claimed is:

1. A biometric detection device mounted on a vehicle, comprising:
at least one radar configured to transmit pulse waves at a predetermined frequency and to receive reflected waves of the pulse waves; and
at least one of (i) a circuit and (ii) a processor with a memory storing computer program code executable by the processor, the at least one of the circuit and the processor configured to cause the biometric detection device to detect a presence or absence of a biological entity inside the vehicle based on received signals of the radar, wherein
the radar is configured to perform transmission and reception multiple times at predetermined intervals,
a reception time of the reflected waves is longer than a time corresponding to a distance between the radar and a position at an end of the vehicle farthest from the radar, and
the at least one of the circuit and the processor further configured to cause the biometric detection device to detect the presence or absence of a biological entity based on the received signals from multiple transmissions and receptions.

2. The biometric detection device according to claim 1, wherein
the at least one of the circuit and the processor further configured to cause the biometric detection device to detect the presence or absence of the biological entity based on temporal variations in the received signals from multiple transmissions and receptions.

3. The biometric detection device according to claim 1, wherein
the at least one of the circuit and the processor further configured to cause the biometric detection device to detect the presence or absence of a biological entity based on frequency characteristics of the received signals from multiple transmissions and receptions.

4. The biometric detection device according to claim 2, wherein
the at least one of the circuit and the processor further configured to cause the biometric detection device to detect the presence or absence of a biological entity based on frequency characteristics of the received signals from multiple transmissions and receptions after detecting the presence or absence of a biological entity based on the temporal variations.

5. The biometric detection device according to claim 1, wherein
the at least one of the circuit and the processor further configured to cause the biometric detection device to:
calculate a relative velocity of a moving object based on any of the received signals; and
determine whether a moving object is present outside the vehicle based on the relative velocity.

6. The biometric detection device according to claim 1, wherein
the radar includes an in-vehicle radar disposed inside the vehicle and an out-of-vehicle radar disposed outside the vehicle, and
the at least one of the circuit and the processor further configured to cause the biometric detection device to detect the presence or absence of a biological entity based on received signals of the in-vehicle radar and the out-of-vehicle radar.

7. The biometric detection device according to claim 1, comprising:
radars disposed inside the vehicle, wherein
the radar is one of the radars,
the radars are configured to perform transmission and reception multiple times at mutually different times, and
the at least one of the circuit and the processor further configured to cause the biometric detection device to detect the presence or absence of a biological entity based on the received signals from multiple transmissions and receptions of each radar.

8. The biometric detection device according to claim 1, wherein
the radar is an ultra-wideband radar.

9. The biometric detection device according to claim 1, wherein
the at least one of the circuit and the processor further configured to cause the biometric detection device to detect the presence or absence of the biological entity based on temporal variations in reception strength at multiple sampling points of the received signals from multiple transmissions and receptions, the sampling points including points where indirect waves reflected from the biological entity inside the vehicle are observable.
